(19) United States
(12) Patent Application Publication
(10) Pub. No.: US 2025/0173771 A1
(43) Pub. Date: May 29, 2025

(54) METHODS AND SYSTEMS FOR CUSTOMIZATION OF COSMECEUTICALS

(71) Applicant: MEDISCA PHARMACEUTIQUE INC., Saint-Laurent (CA)

(72) Inventors: Panagiota DANOPOULOS, Saint-Laurent (CA); Fanny DUBOIS, Saint-Laurent (CA); Amanda CLARIDGE, Saint-Laurent (CA)

(73) Assignee: MEDISCA PHARMACEUTIQUE INC., Saint-Laurent, QC (CA)

(21) Appl. No.: 19/040,110

(22) Filed: Jan. 29, 2025

Related U.S. Application Data
(63) Continuation of application No. 17/613,666, filed on Nov. 23, 2021, now abandoned.
(60) Provisional application No. 62/851,825, filed on May 23, 2019.

(51) Int. Cl. G06Q 30/0601; A45D 44/00

(52) U.S. Cl. CPC G06Q 30/0621; A45D 2044/007

(57) ABSTRACT

A computerized system for initiating preparation of a customized cosmeceutical formulation, comprising a processor configured to execute program instructions to instantiate a cosmeceutical utility configured for (i) obtaining a skin type, a first skin concern and a prioritized set of second skin concerns from a user; (ii) selecting a base composition from a set of base compositions based on the skin type and the first skin concern; (iii) selecting a subset of booster compositions by determining a prioritized original set of booster compositions based on the prioritized set of second skin concerns; and verifying mutual compatibility of a subset of booster compositions in the original set. If the verifying indicates incompatibility among two or more booster compositions in the subset, the utility is configured to substitute a lower-priority booster composition in the two or more booster compositions with a booster composition that is in the original set but not in the subset; and (iv) causing an electronic message comprising a request to be released towards a recipient, the request identifying the selected base composition and the selected subset of booster compositions.

### CROSS-REFERENCE TO RELATED APPLICATION

The present application claims the benefit of U.S. Provisional Application 62/851,825, filed on May 23, 2019, hereby incorporated by reference herein.

### FIELD

The present document relates generally to compounding of cosmeceuticals and, in particular, to new methods and systems for customizing such cosmeceuticals for different individuals according to their specific skincare or haircare situation.

### BACKGROUND

Cosmetic products such as creams, gels and lotions having, or purported to have, beneficial properties (i.e., cosmeceuticals) are used extensively throughout the world. Typically, a line of cosmeceuticals is made by a particular manufacturer and sold to customers via a point of sale. However, individuals have different skin types and skin concerns. As such, it is unrealistic to expect that a relatively small number of pre-made cosmetic products will be effective for a wide range of skincare profiles. It would therefore be beneficial if cosmeceutical formulations could be customized and made available to individuals in a convenient way.

### SUMMARY

According to a first broad aspect, there is provided a computerized system for initiating preparation of a customized cosmeceutical formulation. The computerized system comprises: at least one computer processor; and a non-transitory storage medium operably connected to the at least one processor and storing computer-readable program instructions; the at least one processor being configured to execute the program instructions; wherein execution of the program instructions by the at least one processor causes instantiation of a cosmeceutical utility. The cosmeceutical utility is configured for: obtaining a skin type, a first skin concern and a prioritized set of second skin concerns from a user; selecting a base composition from a set of base compositions based on the skin type and the first skin concern; selecting a subset of booster compositions by: determining a prioritized original set of booster compositions based on the prioritized set of second skin concerns; and verifying mutual compatibility of a subset of booster compositions in the original set and, if the verifying indicates incompatibility among two or more booster compositions in the subset, substituting a lower-priority booster composition in the two or more booster compositions with a booster composition that is in the original set but not in the subset; causing an electronic message comprising a request to prepare a cosmeceutical formulation to be released towards a recipient, the request identifying the selected base composition and the selected subset of booster compositions.

According to another broad aspect, there is provided a computer-implemented method. The computer-implemented method comprises: obtaining a skin type, a first skin concern and a prioritized set of second skin concerns from a user; selecting a base composition from a set of base compositions based on the skin type and the first skin concern; selecting a subset of booster compositions by: determining a prioritized original set of booster compositions based on the prioritized set of second skin concerns; and verifying mutual compatibility of a subset of booster compositions in the original set and, if the verifying reveals incompatibility among two or more booster compositions in the subset, substituting a lower-priority booster composition in the two or more booster compositions with a booster composition that is in the original set but not in the subset; causing an electronic message comprising a request to prepare a cosmeceutical formulation to be released towards a recipient, the request identifying the selected base composition and the selected subset of booster compositions.

According to another broad aspect, there is provided a computerized system for initiating preparation of a customized cosmeceutical formulation. The computerized system comprises: at least one computer processor; and a non-transitory storage medium operably connected to the at least one processor and storing computer-readable program instructions; the at least one processor being configured to execute the program instructions; wherein execution of the program instructions by the at least one processor causes instantiation of a cosmeceutical utility. The cosmeceutical utility is configured for: implementing a graphical user interface configured for interactively eliciting a personal care profile from a user; obtaining at least one ingredient requirement; generating a formula for producing a cosmeceutical formulation based on the user's personal care profile and the at least one ingredient requirement; and causing an electronic message comprising a request to prepare the cosmeceutical formulation to be released towards a recipient, the request specifying the generated formula.

According to another broad aspect, there is provided a cosmeceutical services method, comprising: interacting with a graphical user interface (GUI) of a communication device to provide a personal care profile and a target ingredient to a computing device over a data network; receiving over a merchandise delivery network a customized cosmeceutical formulation produced by a compounding facility in accordance with a formula generated by the computing device based on at least partly on the personal care profile, the customized cosmeceutical formulation comprising the target ingredient.

According to another broad aspect, there is provided a system for initiating preparation of a customized cosmeceutical formulation. The system comprises: at least one computer processor; and a non-transitory storage medium operably connected to the at least one processor and storing computer-readable program instructions; the at least one processor being configured to execute the program instructions; wherein execution of the program instructions by the at least one processor causes instantiation of a cosmeceutical utility. The cosmeceutical utility is configured for: implementing a graphical user interface configured for interactively eliciting a personal care profile from a user; obtaining expiry date information regarding each of a plurality of batches of ingredients; generating a formula for producing a cosmeceutical formulation based on the user's personal care profile and the expiry date information; and causing an electronic message comprising a request to prepare the cosmeceutical formulation to be released towards a recipient, the request comprising the generated formula.

According to another broad aspect, there is provided a method, comprising: implementing a graphical user interface configured for interactively eliciting a personal care profile from a user; obtaining expiry date information regarding each of a plurality of batches of ingredients; generating a formula for producing a cosmeceutical formulation based on the user's personal care profile and the expiry date information; causing an electronic message comprising the generated formula towards a recipient, the request comprising the generated formula; and at the recipient, receiving the generated formula and compounding a cosmeceutical formulation according to the generated formula.

According to another broad aspect, there is provided a method, comprising: obtaining a skin type, a first skin concern and a prioritized set of second skin concerns from a user; selecting a base composition from a set of base compositions based on the skin type and the first skin concern; selecting a subset of booster compositions by: determining a prioritized original set of booster compositions based on the prioritized set of second skin concerns; and verifying mutual compatibility of a subset of booster compositions in the original set and, if the verifying reveals incompatibility among two or more booster compositions in the subset, substituting a lower-priority booster composition in the two or more booster compositions with a booster composition that is in the original set but not in the subset; and preparing the cosmeceutical formulation based on the selected base composition and the selected subset of booster compositions.

These and other aspects of this disclosure will now become apparent to those of ordinary skill upon review of a description of embodiments that follows in conjunction with accompanying drawings.

### BRIEF DESCRIPTION OF THE DRAWINGS

Embodiments will be described, by way of example only, with reference to the accompanying figures wherein:

FIG. 1 shows a high-level block diagram of the connection between a communication device, a web server and a compounding facility, according to a non-limiting embodiment;

FIG. 2A shows inner workings of a computing device such as the communication device, according to a non-limiting embodiment;

FIG. 2B shows inner workings of the computing device such as the web server, according to a non-limiting embodiment;

FIG. 2C is a schematic view of a memory element representing a memory of the web server and a memory of the communication device, according to a non-limiting embodiment;

FIG. 3 shows an embodiment of a cosmeceutical utility that comprises a formula rules engine and a control module, according to a non-limiting embodiment;

FIG. 4 is a diagram illustrating a non limiting example logic behind administering a questionnaire, according to a non-limiting embodiment;

FIG. 5A is a flowchart showing example operation of the formula rules engine in the selection of a single base, according to a non-limiting embodiment;

FIG. 5B shows a variant of one of the steps in the flowchart of FIG. 5A, according to a non-limiting embodiment;

FIG. 6A shows a non-limiting example of an enhancement to operation of the cosmeceutical utility in which the control module is configured to request and acquire additional input from a user;

FIG. 6B shows the control module configured to request and obtain additional input from a medical record processing facility, according to a non-limiting embodiment;

FIG. 7 is a flowchart showing example operation of the formula rules engine processing an indication of at least one target ingredient, according to a non-limiting embodiment;

FIG. 8 shows a non-limiting example of a database of bases;

FIG. 9 shows a non-limiting example of a database of boosters;

FIG. 10 shows a networked computer at the compounding facility including a database conceptually maintaining a record of quantities and expiry dates of various products, according to a non-limiting embodiment;

FIG. 11 shows a non-limiting example of a personal care profile for a user;

FIG. 12A is a block diagram of a compounding facility showing a video camera with an internet feed providing live images to a user wishing to see a formulation being compounded at the compounding facility, according to a non-limiting embodiment;

FIG. 12B shows a progress bar displayed on a screen of the communication device in such a way as to show a current stage of production, according to a non-limiting embodiment;

FIGS. 13 to 16 are non-limiting flowcharts showing example operation of various computer-implemented methods.

It is to be expressly understood that the description and drawings are only for purposes of illustrating certain embodiments and are an aid for understanding. They are not intended to be and should not be limiting.

### DETAILED DESCRIPTION

In the present description, a customer may be interested in purchasing a skincare product or a haircare product, with the aim being alignment with the customer's personal skincare profile (e.g., skin type, skin condition and/or skin concerns) or haircare profile (e.g., hair type, hair condition and/or hair concerns).

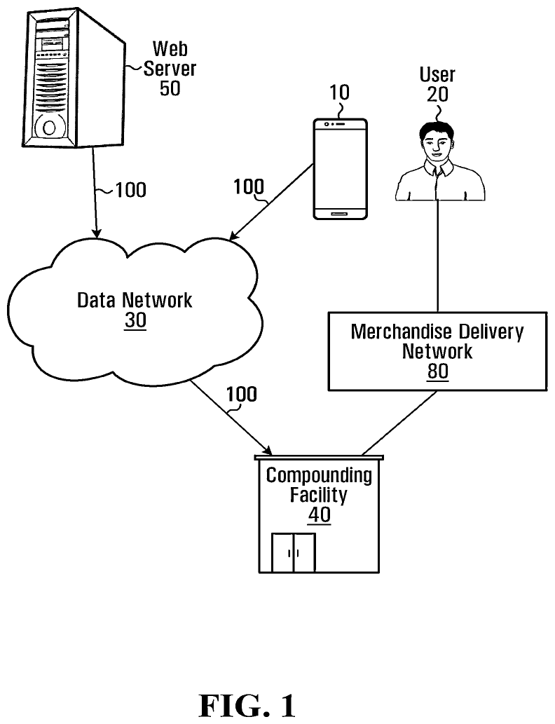

Accordingly, and with reference to FIG. 1, there is shown a communication device 10 (e.g., a smartphone, laptop, PC, etc.) and a user 20 of the communication device 10. Also shown is a compounding facility 40 (e.g., a licensed compounding pharmacy), which is equipped with a production facility for producing cosmeceutical formulations. The communication device 10 and a communication device (not shown) at the compounding facility 40 are connected to a web server 50 via a data network 30 (e.g., the internet). This connection may involve wireless and/or wireline communication, allowing the exchange of electronic messages among the communication device 10, the compounding facility 40 and the web server 50. Each of the communication device 10, the compounding facility 40 and the web server 50 may therefore execute compatible software or apps to allow the exchange of such messages. The compounding facility 40 and the user 20 are also served by a merchandise delivery network 80 (e.g., public postal service or private messenger service). This allows the compounding facility 40 to physically send prepared formulations to an address associated with the user 20.

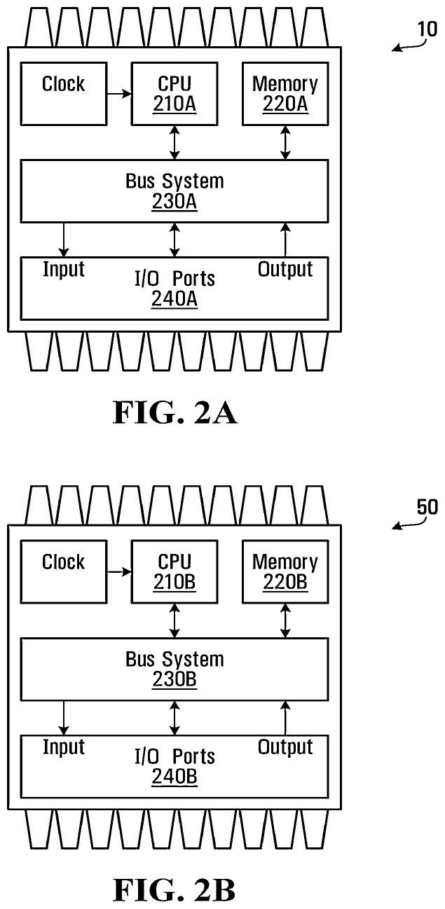

FIG. 2A shows in general the inner workings of a computing device such as the web server 50 and/or the communication device 10, wherein the suffix A denotes a component of the communication device 10 and the suffix B denotes a component of the web server 50. There is provided at least one processor 210A, 210B (e.g., CPU) operably connected via a bus 230A, 230B to a non-transitory storage medium (e.g., non-volatile and/or volatile memory 220A, 220B). The memory 220A, 220B stores computer-readable program instructions. The at least one processor 210A, 210B is configured to execute the program instructions stored in the memory 220A, 220B. Also provided is an I/O 240A, 240B connected to the bus 230A, 230B. The I/O 240A, 240B allows the at least one processor 210A, 210B to communicate with entities outside the computing device. The I/O 240A, 240B may include a user interface (e.g., for connection to a touchscreen, loudspeaker, keyboard, etc.) and a network interface (e.g., an antenna, a network interface card, etc., for providing a connection to the data network 30).

With reference to FIG. 2B, the web server 50 and the communication device 10 cooperate to instantiate a cosmeceutical utility 280 for interacting with the user 20 and ultimately sending a cosmeceutical formula to the compounding facility 40 over the data network 30. Specifically, execution of program instructions stored in the memory 220B of the web server 50 and/or in the memory 220A the communication device 10 causes instantiation of the cosmeceutical utility 280. The cosmeceutical utility 280 may be based on a web model or an app model. In the case of a web model, the cosmeceutical utility 280 may execute primarily on the web server 50, using the communication device 10 merely as a conduit for presenting and gathering information. In the case of an app model, the cosmeceutical utility 280 may execute partly on the communication device 10 and partly on the web server 50. As such, the program instructions are shown as being part of a memory object denoted 220A/220B, which conceptually represents both the memory 220A of the communication device 10 and the memory 220B of the web server 50.

Similarly, the data elements used by the cosmeceutical utility are also stored in the memory object 220A/220B. These data elements include a questionnaire 292, a personal care profile database 293, a database of bases 294, a database of boosters 295, a booster compatibility table 296 and a tested formula database 297. Each of these data elements will be described in further detail herein below. It should be appreciated that not all data elements are required in all embodiments.

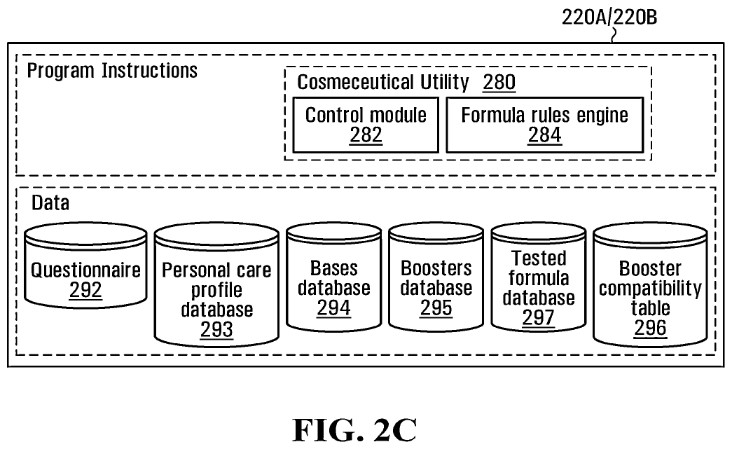

In an embodiment, the cosmeceutical utility 280 includes one or more cooperating functional modules, such as a control module 282 and a formula rules engine 284. Operation of the cosmeceutical utility 280 and interaction of the control module 282 and formula rules engine 284 will best be understood with reference to the flow diagram in FIG. 3, which illustrates steps executed by the cosmeceutical utility 280.

First, the cosmeceutical utility 280 invokes the control module 282. Generally speaking, the control module 282 is configured for implementing a graphical user interface (GUI) and interactively eliciting a personal care profile from the user 20 via such graphical user interface (step 310). Interactively eliciting a personal care profile can be done by administering a questionnaire 292 stored in the memory 220A or 220B. For example, administering the questionnaire 292 may include providing a first set of options to the user 20 via the GUI, collecting the user's selection of an option from the first set of options via the GUI, and interactively providing a second set of options to the user 20 via the GUI, wherein the second set of options may depend on the option from the first set of options that had been selected by the user 20. This can be done for numerous sets of options in sequence, so as to elicit a particular set of selections for particular sets of options. These selections can be said to define to the personal care profile of the user 20, which may then be stored in a personal care profile database 293 in association with an identifier of the user 20. By way of non-limiting example, a personal care profile may include type, condition and/or concerns regarding the user's skin and/or hair, to name a few non-limiting possibilities.

The control module 282 may optionally also be configured for acquiring an image from a camera of the communication device 10 (step 320). This image may be of the user's face or head, for example, and may also be stored in memory 220A or 220B. An image acquisition device (e.g., CCD camera) mounted to the communication device 10 may be used for this purpose. Operation of the camera may be controlled by the user 20 of the communication device 10 or it may be automatically controlled by the web server 50.

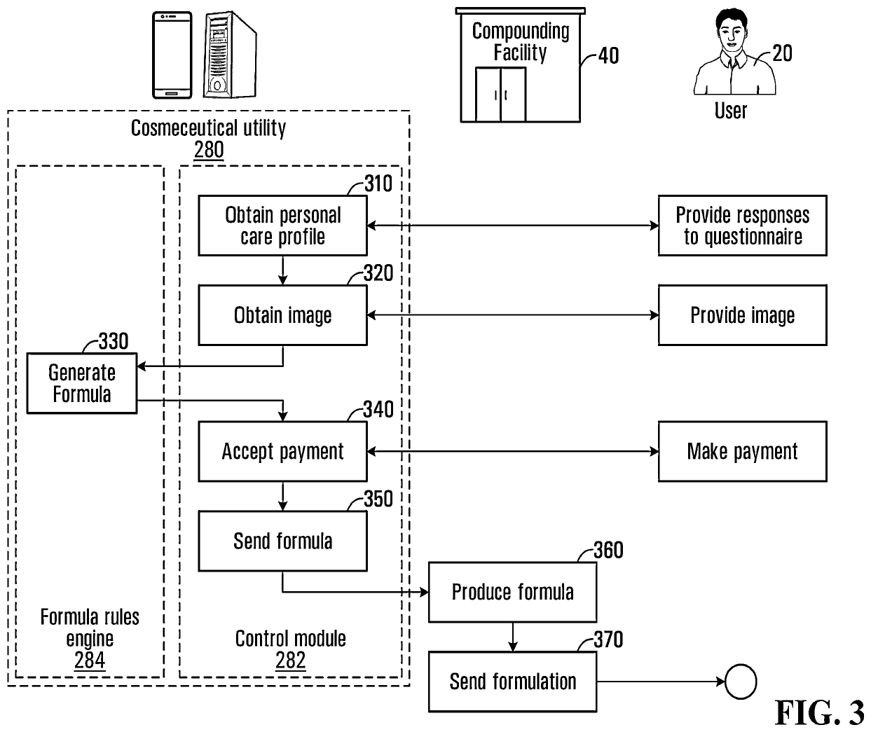

FIG. 4 shows a diagram illustrating example logic behind administering the questionnaire 292, thereby to elicit the user's personal care profile. In particular, the user 20 is first asked a set of questions to ascertain the user's skin type along the dryness-oiliness spectrum (e.g., “very dry”, “dry”, “normal”, “oily/dry”, “oily” or “very oily”).

The user 20 is also asked a set of questions allowing the control module 282 to ascertain a Category 1 skin concern (which may sometimes be referred to as a first skin concern) and a plurality of Category 2 skin concerns (which may sometimes be referred to as a plurality of second skin concerns). Examples of Category 1 skin concerns may be based on the user's perceived problems with their skin and may include, for example, “aging”, “post-procedure recovery”, “moisture”, “sensitivity” and “oily skin/blemishes”. In some cases, the possible Category 1 skin concerns may be directly selectable by the user. In other cases, the possible Category 1 skin concerns may be dependent on (e.g., restricted by) the skin type. For example, if the user's skin type was found to be “oily”, then it is possible that the Category 1 skin concern is limited to one of “aging”, “post-procedure recovery”, “moisture”, “sensitivity” and “oily skin/blemishes”, whereas if the user's skin type was found to be “very oily”, perhaps the only possible Category 1 skin concern will be “oily skin/blemishes”.

As for the Category 2 skin concerns, these also ascertained based on more in-depth questions asked of the user 20 (e.g., as to the user's perception of the severity of his or her skin problems), and are dependent to a certain extent on the Category 1 skin concern. For example, as shown in FIG. 4, if the Category 1 skin concern is “aging”, then the Category 2 skin concerns can be “wrinkles”, “instant wrinkle filling”, “sun damage”, “dark spots”, “excess oil/blemishes”, “redness”, “general sensitivity”, “Botox®, . . . ”, “fillers”, “laser” and “no procedure”. On the other hand, if the Category 1 skin concern is “moisture”, then the Category 2 skin concerns can be “wrinkles”, “excess oil, blemishes”, dehydration” and “dryness”. Moreover, an ordering of the Category 2 skin concerns (e.g., in decreasing level of importance to the user) may be ascertained based on the answers received from the user 20. That is to say, in cases where the user 20 selects more than one Category 2 skin concern, the control module 282 may be configured to allow the user 20 to rank the selected Category 2 skin concerns in order of importance.

The aforementioned Category 1 and Category 2 skin concerns may be referred to as “primary” skin concerns and in some embodiments, the control module 282 may also be configured to ascertain a set of “secondary” skin concerns, namely a secondary Category 1 skin concern and a plurality of secondary Category 2 skin concerns. The options are basically the same as for the primary Category 1 and Category 2 skin concerns, except with the restriction that the secondary Category 1 skin concern must be different from the primary Category 1 skin concern. It is noted that in some cases, the secondary Category 2 skin concerns may end up having some commonality with the primary Category 2 skin concerns. For example, “general sensitivity” as a Category 2 skin concern may be reached through a Category 1 skin concern that is either “aging” or “sensitivity”. In the case of very oily skin, however, as the primary Category 1 skin concern was restricted to just a single option (namely, “oily skin/blemishes”), there is no available secondary Category 1 skin concern.

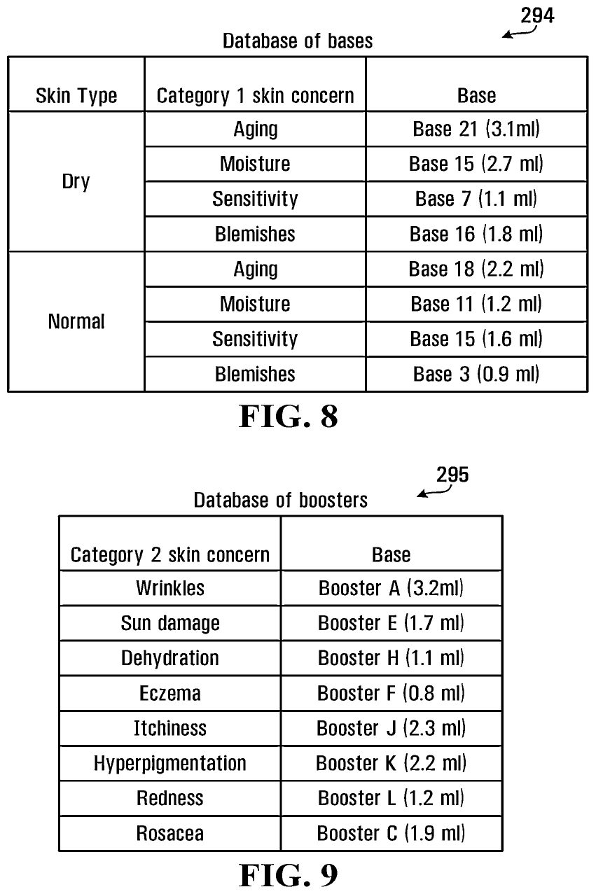

In this non-limiting example, the skin type, primary Category 1 and 2 skin concerns and, if applicable, secondary Category 1 and 2 skin concerns, make up the personal care profile for the user 20. The personal care profile for the user 20 may be stored in the personal care profile database 293, e.g., in the form of a record associated with an identifier of the user 20. With reference to FIG. 11, there is shown an example personal care profile database 293, including a skincare record 1110 for each user, and each skincare record including a plurality of fields, including:
User ID 1120 (e.g., name, phone number, etc.)
Primary Category 1 skin concern 1130
Primary Category 2 skin concern(s) 1140
Secondary Category 1 skin concern (if applicable) 1150
Secondary Category 2 skin concern(s) (if applicable) 1160

Other optional fields may also exist (not shown for simplicity):
Name, code and/or address of preferred pharmacy
Name, code and/or address of healthcare provider
Medications/prescriptions
Allergies
Conditions

Similar records may be created for haircare.

Having acquired or accessed the personal care profile, the cosmeceutical utility 280 is then configured to invoke the formula rules engine 284. Accordingly, and returning now to FIG. 3 (step 330), the formula rules engine 284 is configured for generating a formula (i.e., a cosmeceutical formula) based on the user's personal care profile (received at step 310) and possibly also on the image acquired by the camera (at step 320). The generated formula will ultimately be sent to and used by the compounding facility 40 for producing a customized cosmeceutical formulation for the user 20.

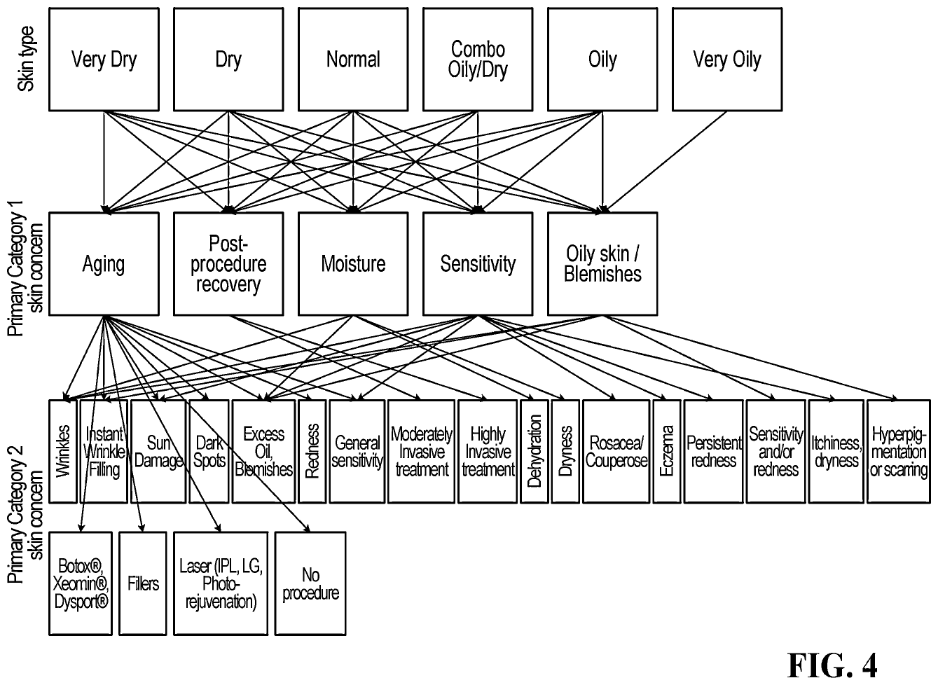

FIG. 5A shows a flowchart defining example operation of the formula rules engine 284. In this example, the personal care profile is a skincare profile and includes a skin type, a primary Category 1 skin concern (hereinafter simply a Category 1 skin concern) and up to N primary Category 2 skin concerns (hereinafter simply Category 2 skin concerns) related to the Category 1 skin concern, where N can be 1, 2, 3 or more. The personal skincare profile is for the time being assumed not to include a secondary set of skin concerns, although this possibility is described later on.

At the output end, a generated formula is output by the formula rules engine 284 and this generated formula includes a base composition (or “cosmetic base”, or simply “base”) selected from the database of bases 294, in addition to M booster compositions (or “boosters”) selected from the database of boosters 295, where M can be 1, 2, 3 or more. It is noted that M (the selected number of boosters) may, but need not be, the same as N (the number of primary Category 2 skin concerns), i.e., there may, but need not be, the same number of selected boosters in the final formula as there are primary Category 2 skin concerns.

The bases and boosters are compositions with respective lists of ingredients. Non-limiting examples of bases and boosters can be found in U.S. Pat. No. 9,522,112 to Medisca Pharmaceutique Inc., hereby incorporated by reference herein. The boosters include cosmetic active ingredients which, when mixed with a chosen cosmetic base, may boost effectiveness or performance of the chosen cosmetic base to address the primary skin concerns and/or the secondary skin concerns of the user 20 or achieve specific benefits.

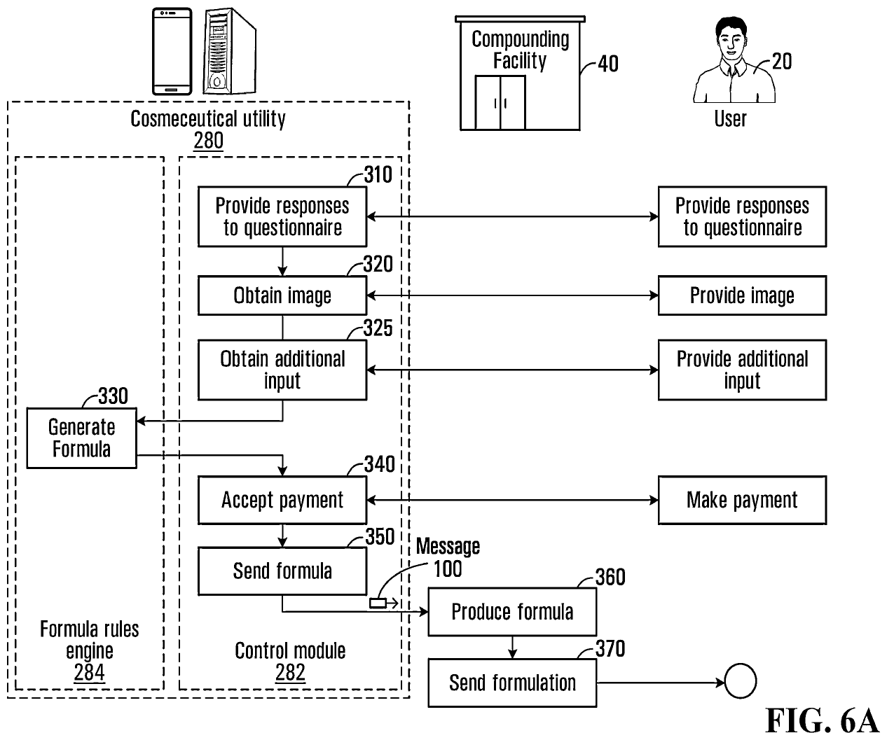

Step 510: The formula rules engine 284 is configured to consult the database of bases 294 and to select a base as a function of, e.g., the skin type and Category 1 skin concern. The association between skin type and Category 1 skin concern on the one hand, and base on the other, may be stored in the database of bases 294. Specifically, FIG. 8 shows the database of bases 294 illustrated as a table 800 that associates skin types and Category 1 skin concerns to bases (potentially also including corresponding dosage amounts). It is possible that some bases are commonly used for different combinations of skin type and Category 1 skin concern. In this example, the same base (Base 15) is used for addressing “moisture” as a Category 1 skin concern for the “dry” skin type and for addressing “sensitivity” as a Category 1 skin concern for the “normal” skin type (albeit in different dosages). Of course, it should be understood that a table is used for convenience of illustration and that other data structures may be employed.

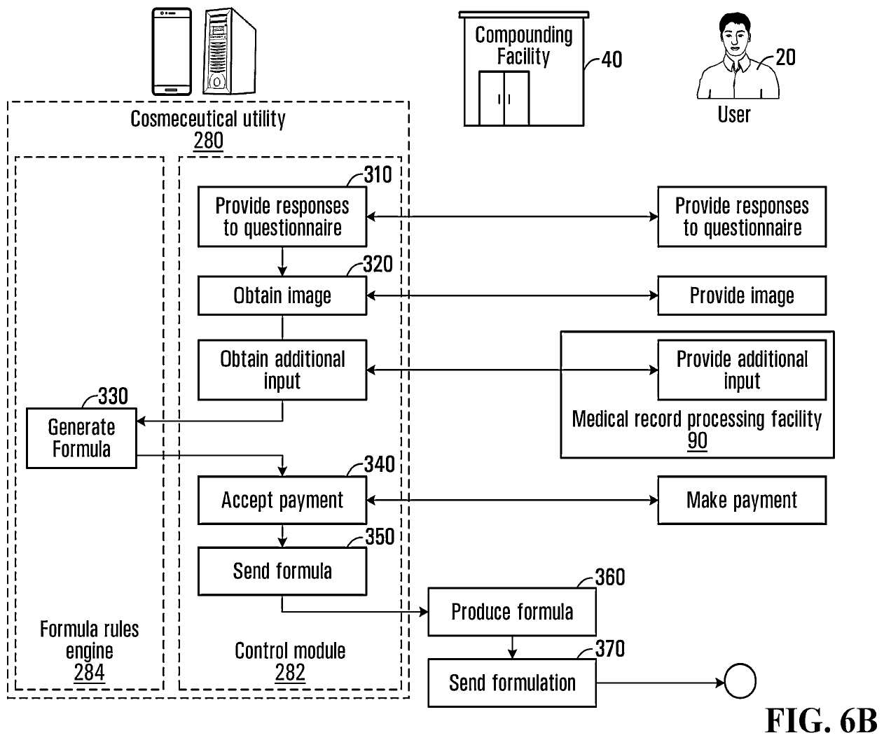

Step 520: The formula rules engine 284 is configured to populate an array of candidate boosters 500, based on, e.g., the Category 2 skin concerns. An association between Category 2 skin concerns and boosters (in corresponding dosage amounts) may be stored in the database of boosters 295. Specifically, FIG. 9 shows the database of boosters 295 illustrated as a table that associates each Category 2 skin concern to a booster (potentially also including a corresponding dosage amount). Of course, it should be understood that a table is used for convenience of illustration and that other data structures may be employed. It should also be noted that some boosters may be used repeatedly (albeit in different dosages) for different Category 2 skin concerns.

In the case where each Category 2 skin concern is associated with a single booster, as depicted in the non-limiting example of FIG. 9, this leads to the array of candidate boosters 500 being populated with as many candidate boosters as there are Category 2 skin concerns, i.e., the array of candidate boosters 500 will have N entries. The array of candidate boosters 500 may be populated in an order that corresponds to the order in which the Category 2 concerns appear in the personal skincare profile, as selected by the user 20 in terms of decreasing order of importance.

However, in some cases, one or more of the Category 2 skin concerns may be associated with more than just one booster. For example, if the Category 1 skin concern was “oily skin”, then this may present a set of options for the Category 2 skin concerns including “excess oil”, “hyperpigmentation” and “wrinkles”. It is possible that “hyperpigmentation” and “wrinkles” are each associated to a single (different) booster and that “excess oil” is associated to 2 other boosters. This leads to the array of candidate boosters 500 being populated with potentially more candidate boosters than there are Category 2 skin concerns, i.e., it may have more than N entries. Also, the array of candidate boosters 500 may be populated in an order that corresponds to the order in which the Category 2 concerns appear in the personal skincare profile, as selected by the user 20 in terms of, say, decreasing order of importance.

It should also be noted that if two Category 2 skin concerns are associated to the same booster, the result is a single candidate booster, in a total dosage amount that may, but need not, be the sum of the dosage amounts. In scenarios where the dosage amount of the same candidate booster is the same, then that dosage amount is selected to be the final amount of the candidate booster. However, there are scenarios where the dosage amount of the same candidate booster can be different and in this case, the higher dosage amount could be selected. For example, where the Category 1 skin concern is “sensitivity”, the Category 2 skin concern “general sensitivity-moderate to significant” may be associated with a candidate booster with a dosage amount of 2.6 ml (5%) and the Category 2 skin concern “wrinkles-grade 1” may be associated with the same candidate booster but with a dosage amount of 1.1 ml (2%). If both “general sensitivity-moderate to significant” and “wrinkles-grade 1” are selected as Category 2 skin concerns, the higher dosage amount (2.6 ml) for the candidate booster could be selected. In still other embodiments, the average could be selected.

Step 530: The formula rules engine 284 is configured to determine a maximum number of boosters permitted in the formula to be generated. The maximum number of boosters may be dependent on factors such as the Category 1 skin concern and the Category 2 skin concern(s). For example, if the Category 1 skin concern is “aging” or “moisture”, then the number of boosters permitted in the formula may be limited to 2. On the other hand, if the Category 1 skin concern is “sensitivity”, then the number of boosters in the formula may be limited to 3. Still further, if the Category 1 skin concern is “oily skin”, then the number of boosters in the formula may depend on which Category 2 skin concerns were selected by the user. That is to say, for example, certain Category 2 skin concerns, if selected by the user, would limit the number of boosters to 2 and certain other Category 2 skin concerns, if selected by the user, would limit the number of boosters to 3.

Step 540: If the array of candidate boosters 500 (see step 520) has more than the maximum number of boosters permitted in the formula (as determined at step 530), then the number of candidate boosters in the array 500 is reduced to such maximum number. By selecting candidate boosters that are at the “beginning” of the list and not selecting candidate boosters that are at the “end” of the list, the formula rules engine 284 ends up selecting those candidate boosters that are relevant to the Category 2 skin concerns that were prioritized by the user 20. Depending on the embodiment, up to 2 or 3 (or more) candidate boosters in the array may end up being selected. Unselected boosters are retained in memory but not considered “selected”.

Step 550: The computing device running the formula rules engine 284 may be configured to verify mutual compatibility of the selected boosters. To this end, a booster compatibility table 296 may be consulted. The booster compatibility table 296 may list pairs (or groups) of boosters that are compatible with one another and/or pairs (or groups) of boosters that are not compatible with one another, depending on the embodiment.

For example, assume that the array of candidate boosters 500 included 4 boosters A, B, C and D. Depending on the Category 1 skin concern, the maximum number of boosters may be just 2. This could mean that boosters A and B form a subset of selected boosters after execution of step 540. As such, at step 550, the formula rules engine 284 consults the booster compatibility table 296 to verify whether booster A is compatible with booster B. If yes, then compatibility verification has successfully terminated and the formula rules engine 284 proceeds to step 560.

However, if booster A is determined to be not compatible with booster B, then the formula rules engine 284 replaces booster B (because booster B is lower on the priority list than booster A) with booster C (the next booster in the array of candidate boosters 500, but which was up until now not considered “selected”) and proceeds to verify compatibility between booster A and booster C. If compatibility verification is still not successful, then the exercise is repeated for booster A and booster D.

As such, if verifying reveals incompatibility among two or more boosters in the subset of selected boosters, then the computing device substitutes a lower-priority booster among these two or more boosters with a booster that is in the array of candidate boosters but not in the subset of selected boosters.

Once compatibility verification by the computing device has successfully terminated, this yields a final subset or combination of boosters (up to the maximum number) and dosage amounts which, combined with the selected base (see step 510), defines a candidate formula. The formula rules engine 284 then proceeds to step 560.

Step 560: The formula rules engine 284 may be configured verify if the candidate formula corresponds to a pre-tested formula. To this end, the formula rules engine 284 may be configured to access the tested formulas database 297 in order to verify whether the candidate formula appears in the tested formulas database. If yes, the formula rules engine terminates 284 and the candidate formula becomes the generated formula. If not, the formula rules engine 284 proceeds to step 570. The tested formulas base 297 may be a dynamically updated list of formulas that have been tested for, e.g., safety or compatibility with human skin. This list may be updated on the web server 50 by the compounding facility 40 over the data network 30.

Step 570: In the event that step 560 reveals that the candidate formula is not a pre-tested formula (e.g., does not appear in the tested formulas database 297), the formula rules engine 284 may be configured to select a default set of boosters based on the skin type and the Category 1 skin concern. For example, if the skin type is “normal” and the Category 1 skin concern is “aging”, the default formula may include a pre-tested combination of a suitable base for the skin type and Category 1 skin concern (e.g., a “light anti-aging cream” base) together with zero or more boosters, covering a general set of aging concerns while ignoring the user's Category 2 skin concerns that may would have resulted from the user's interaction with the control module 282. This may lead to a sub-optimal skincare product but may be a suitable alternative when the formula rules engine 284 is unable to find, in the tested formula database 297, a particular formula that matches the candidate formula. The combination of the suitable base with the default set of boosters becomes the generated formula. In other embodiments, the mere fact that the candidate formula is not a pre-tested formula does not impede its output as the generated formula; however, the computing device carrying out the formula rules engine 284 may be configured to issue a signal including a warning message to the user 20 so as to indicate that the generated formula is untested.

In some embodiments, the formula rules engine 284 may produce two or more generated formulas, instead of just one. This may arise when administering the questionnaire results in not just primary Category 1 and Category 2 skin concerns, but also secondary Category 1 and Category 2 skin concerns. In this case, the method of FIG. 5A may be practiced for both the primary Category 1 and Category 2 skin concerns and the secondary Category 1 and Category 2 skin concerns.

For example, FIG. 5B shows step 510*, which is a variant of step 510 in FIG. 5A that resulted in the selection of a single base. Here, step 510* results in the selection of plural candidate bases (e.g., one for the skin type and primary Category 1 skin concern and another one for the skin type and secondary Category 1 skin concern). Again, this could be based on the associations between bases and skin types/conditions stored in the database of bases 294. Each candidate base is then separately run through the remaining steps 520-570 of the formula rules engine 284, resulting in multiple corresponding generated formulas.

A selection step 580 at the very end may be performed by the formula rules engine 284. The selection, which may also include prioritization, could be based on a variety of factors, such as price or ingredient availability. In other embodiments, the selection step 580 may be performed by a doctor, pharmacist or cosmetician based on similar factors or additional factors such as contra-indications based on medical records. The selection may be elicited by the GUI carried out by the cosmeceutical utility 280.

The cosmeceutical utility 280 then proceeds with operation of the control module 282. Specifically, and returning again to FIG. 3, the control module 282 may be configured to accept payment from the user 20 (step 340). Specifically, the control module 282 may be configured for determining a purchase price of the customized cosmeceutical formulation made from the generated formula. The purchase price may be obtained by consulting a price list in memory. The price list may be associated with individual ingredient prices, base prices, booster prices or formulation prices, all on a per-dosage or per-weight basis. The control module 282 may then be configured to interactively elicit payment of the purchase price from the user 20 via the GUI. In some embodiments, payment may be a pre-requisite for shipping or even producing the customized cosmeceutical formulation. In other embodiments, step 340 may be omitted and the user 20 may pay upon delivery of the customized cosmeceutical formulation, either at home or at their local compounding facility 40.

With continued reference to FIGS. 1 and 3, the control module 282 is also configured for causing an electronic message 100 to be released by the web server 50 or the communication device 10 towards a recipient (step 350). The recipient of the message 100 may be the compounding facility 40, which is connected to the web server 50 over the data network 30. The message 100 may comprise a request to prepare a customized cosmeceutical formulation based on the formula generated by the formula rules engine 284, whose operation was described above. Another factor that may affect formation of the message 100 is the type of equipment used by the compounding facility 40. For example, the compounding facility 40 may utilize a planetary mixer of a certain type. The planetary mixer has certain settings for mixing time, mixing speed, rotation: revolution ratio, and so on. A first table may be stored in the memory element 220A/220B that maps each generated formula to specific sets of mixing settings (parameters) optimized for a certain type (e.g., make and model) of mixer, whereas a second table may be stored in the memory element 220A/220B that maps the pharmacy hub 40 to a particular type of mixer. In this way, by knowing the type of mixer at the compounding facility 40, the control module 282 may ascertain the correct set of mixing parameters to be used by the compounding facility 40 in order to result in optimized production of the customized cosmeceutical formulation. This set of mixing parameters may therefore be represented by, or included in, the message 100.

The message 100 may be sent via email, text message or other type of electronic delivery system. In a non-limiting example, the message 100 may be an email message and an email server associated with the web server 50 may communicate with an email server associated with the compounding facility 40, to allow the compounding facility 40 to receive email messages from the web server 50, such as an email message comprising the aforementioned request to prepare a cosmeceutical formulation based on the generated formula.

The message 100 containing the request may be received by a networked computer at the compounding facility 40. Thereafter, the message 100 is read by the networked computer and the generated formula is extracted from the request. A pharmacist or a robot at the compounding facility 40 proceeds to produce the customized cosmeceutical formulation, in accordance with the generated formula (step 360). In an embodiment, the generated formula specifies a base and one or more boosters in corresponding dosage amounts, as discussed earlier in this document. In another embodiment, the message 100 additionally comprises mixing parameters that are extracted by the networked computer and supplied to a mixing device, such as the Maz™. By automating the reading and interpretation of mixing parameters, compounding may be rendered more efficient than is possible with human pharmacists, and human error may be reduced or eliminated at the point of compounding. Once the customized cosmeceutical formulation is prepared, the customized cosmeceutical formulation may be shipped from the compounding facility 40 to the user 20 over the merchandise delivery network 80 to a physical address of the user 20 (step 370).

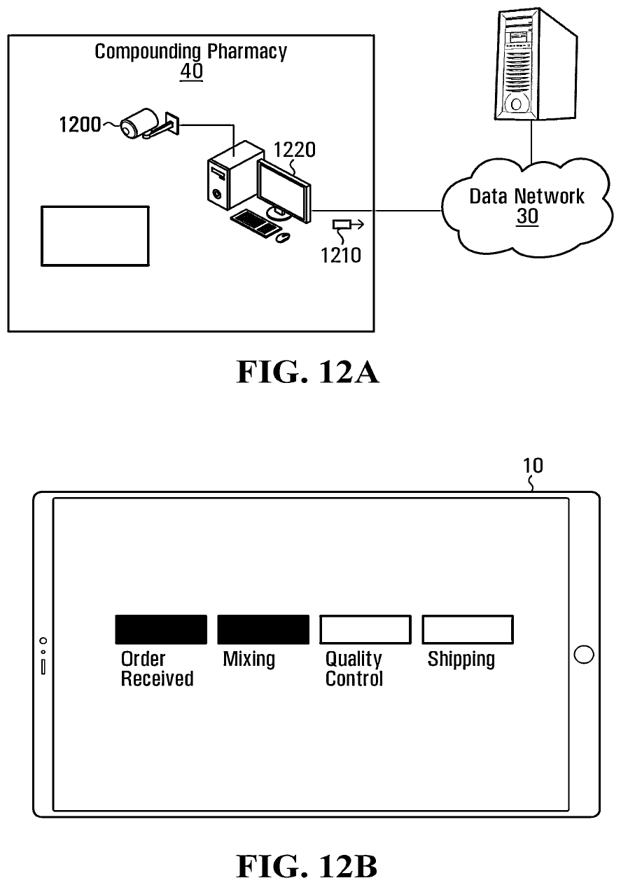

As such, there has been provided a computer-implemented method, which is depicted in the flowchart of FIG. 13 as comprising the steps of obtaining a skin type, a first skin concern and a prioritized set of second skin concerns from a user (step 1310), selecting a base composition from a set of base compositions based on the skin type and the first skin concern (step 1320) and selecting a subset of booster compositions (step 1330). Step 1330 may include determining a prioritized original set of booster compositions based on the prioritized set of second skin concerns (sub-step 1332) and verifying mutual compatibility of a subset of booster compositions in the original set and, if the verifying indicates or reveals incompatibility among two or more booster compositions in the subset, substituting a lower-priority booster composition in the two or more booster compositions with a booster composition that is in the original set but not in the subset (sub-step 1334). Finally, the method includes causing an electronic message comprising a request to prepare a cosmeceutical formulation to be released towards a recipient, the request identifying the selected base composition and the selected subset of booster compositions (step 1340).

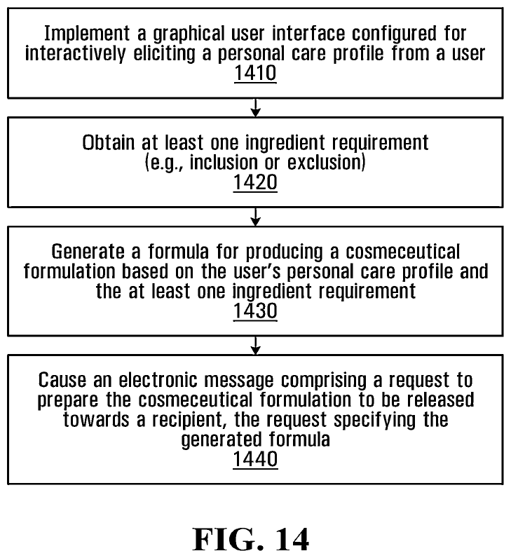

From the user's perspective, a different method is carried out, as depicted in the flowchart of FIG. 15, which includes the steps of interacting with a graphical user interface (GUI) of a communication device (e.g., a smartphone) to provide a personal care profile and a target ingredient to a computing device (e.g., web server) over a data network (step 1510); and receiving over a merchandise delivery network a customized cosmeceutical formulation produced by a compounding facility in accordance with a formula generated by the computing device based on at least partly on the personal care profile, the customized cosmeceutical formulation comprising the target ingredient (step 1520).

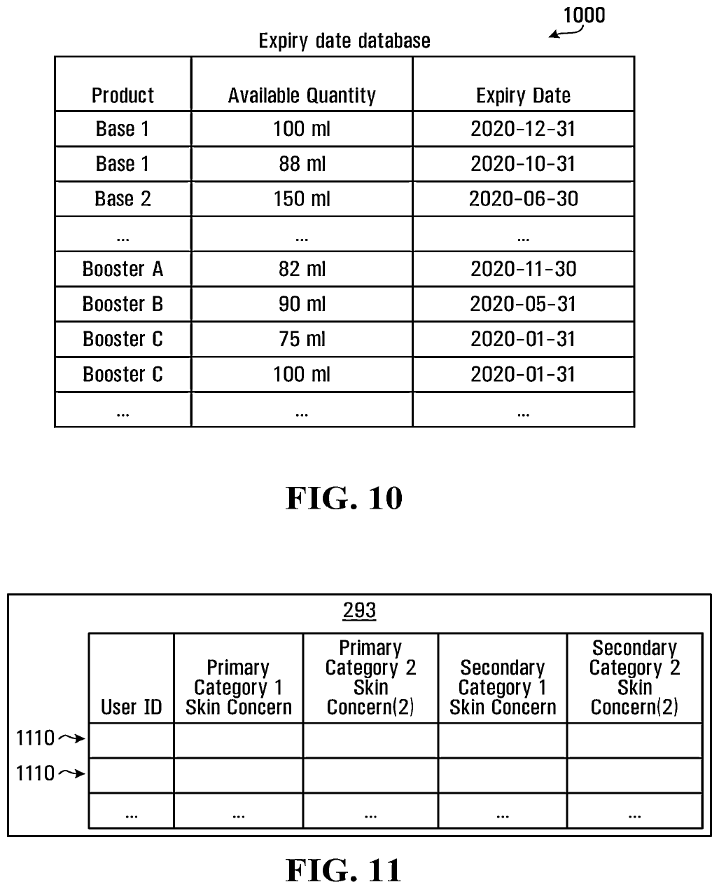

In some embodiments, as depicted in FIG. 12A, a video camera 1200 with an internet feed may provide live images to customers (e.g., the user 20) wishing to see their formulations being compounded at the compounding facility 40. Status messages 1210 can be sent by the compounding facility's networked computer 1220 over the data network 30 to a server 1230 (e.g., a web server) hosting an account of the user 20 to allow the user 20 to keep track of the various stages of production. This information may be provided by a GUI displayed on the communication device 10. For example, as shown in FIG. 12B, a progress bar 1240 may be displayed on a screen of the communication device 10 in such a way as to show the current stage of production (such as “order received”, “mixing”, “quality control”, “shipping”, etc.)

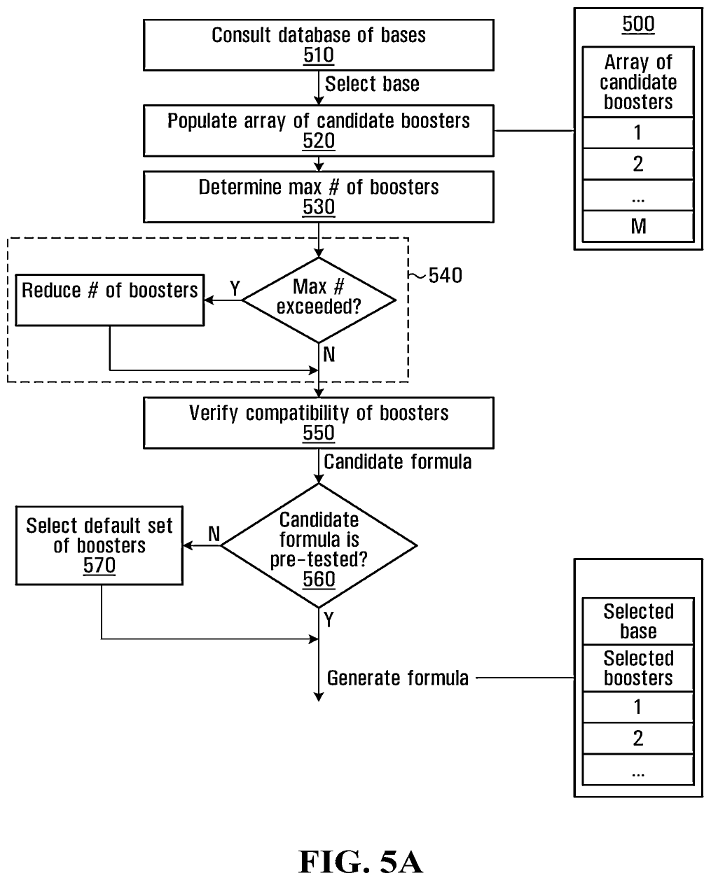

Various enhancements to operation of the cosmeceutical utility 280 are possible. According to a first enhancement, the way in which the generated formula is generated by the formula rules engine 284 is made dependent on “additional input” collected from the user 20. Accordingly, and with reference to FIG. 6A, the control module 282 is configured to request and acquire additional input from the user 20 (step 325).

Such additional input may comprise an indication of a “target ingredient”. In some implementations the target ingredient is an ingredient that is required to be present in the generated formula. In other implementations, the target ingredient is a prohibited ingredient, i.e., one that must be avoided in the generated formula. It should be appreciated that the target ingredient in either of the aforesaid implementations could be a medicinal ingredient or a non-medicinal ingredient. In still further embodiments, more than one target ingredient may be provided, and one or more may be a mandatory ingredient and one or more may be a prohibited ingredient.

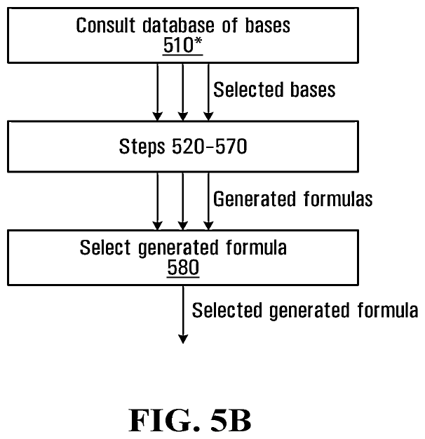

Having acquired the personal care profile and the additional input (e.g., an indication of at least one (required and/or prohibited) target ingredient), the cosmeceutical utility 280 may then be configured to invoke an enhanced version of the formula rules engine 284. The manner in which the enhanced version of the formula rules engine 284 processes the indication of the at least one target ingredient is now described with reference to FIG. 7. Specifically, the formula rules engine 284 may be configured to generate a plurality of formulas, each of which satisfies the user's personal care profile. To this end, an algorithm similar to what was described with reference to FIG. 5B may be used to generate a plurality of formulas each made up of a base and one, two or three boosters, for example. Since each base and each booster has its own set of ingredients, each generated formula can be said to be associated with an ingredient list. The ingredient lists for bases may be stored in the database of bases 294 and the ingredient lists for boosters may be stored in the database of boosters 295, as previously described with reference to FIGS. 8 and 9.

Then, the formula rules engine 284 may be configured to select one of the generated formulas at step 580 (the “current formula”) and, at step 710, determines whether the ingredient list of the current formula satisfies the ingredient criteria. If the ingredient criteria are satisfied for the current formula, the current formula becomes the selected generated formula, which is output to the control module 282 at the conclusion of step 330 (see FIG. 3). For example, if the target ingredient is a mandatory/required ingredient, then the ingredient criteria are satisfied if the ingredient list of the current formula includes the target ingredient. Alternatively, if the target ingredient is a prohibited ingredient, then the ingredient criteria are satisfied if the ingredient list of the current formula excludes the target ingredient.

On the other hand, if it is revealed at step 710 that the ingredient criteria are not satisfied for the current formula, the formula rules engine 284 returns to step 580 and proceeds to select the next one of the generated formulas, and so on, until a generated formula whose ingredient list does satisfy the ingredient criteria is found, in which case that generated formula becomes the “selected” generated formula, which is output to the control module 282 at the conclusion of step 330 (see FIG. 3).

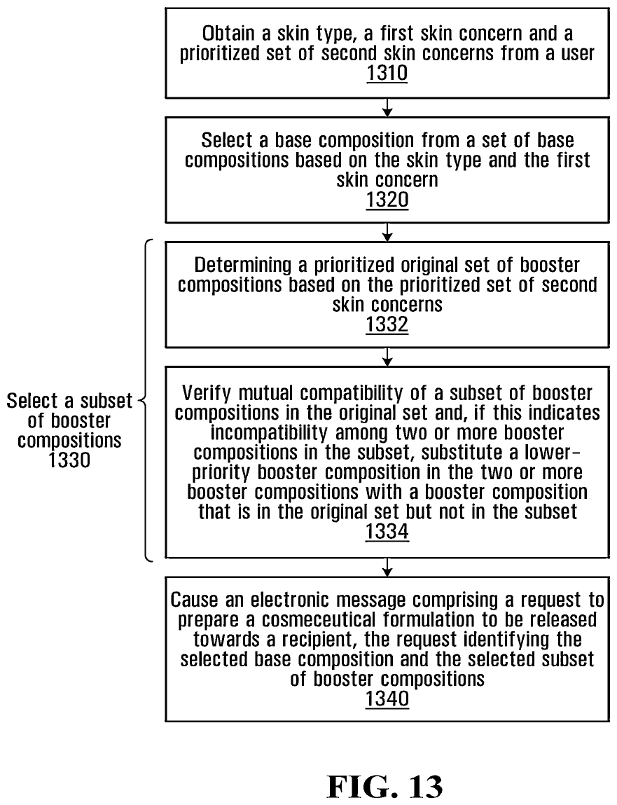

Thus, a cosmeceutical utility has been described. The cosmeceutical utility carries out a method as depicted in the flowchart of FIG. 14, which includes the steps of implementing a graphical user interface configured for interactively eliciting a personal care profile from a user (step 1410); obtaining at least one ingredient requirement (step 1420); generating a formula for producing a cosmeceutical formulation based on the user's personal care profile and the at least one ingredient requirement (step 1430); and causing an electronic message comprising a request to prepare the cosmeceutical formulation to be released towards a recipient, the request specifying the generated formula (step 1440).

In other implementations, the “additional input” received by the control module 282 from the user 20 comprises “medical indicia”, examples of which include a medical condition or a prescription. The control module 282 may thus execute a further step (not shown) that converts the medical indicia into ingredient criteria (such as a target ingredient that is mandatory or prohibited).

For example, where the medical indicia is an allergy to peanuts, the control module 282 determines the ingredients that are to be avoided. Such ingredients may include various proteins known to be contained in peanuts. This leads to the identification of one or more target ingredients that are to be avoided, as checked by the formula rules engine 284 at step 710 of FIG. 7, for example.

In another example, the medical indicia may be a prescription for a certain drug, provided by the user. This is converted by the control module 282 into an active pharmaceutical ingredient (API). Moreover, the prescription may specify a dosage that is to be maintained. This leads to the identification of one or more target ingredients that are mandatory (in certain dosages), as checked by the formula rules engine 284 at step 710 of FIG. 7, for example.

According to a second enhancement, the “additional input” is obtained from an external entity rather than collected from the user 20. Accordingly, and with reference to FIG. 6B, the control module 282 is configured to request and obtain additional input from a medical record processing facility 90. The additional input may include medical indicia (e.g., medical condition or prescription). A link exists between the web server 50 and/or the communication device 10 on the one hand, and the medical record processing facility 90 on the other. The medical record processing facility 90 may comprise a separate server and stores medical records in memory, including a medical record for the user 20 and containing medical indicia (e.g., medical conditions, prescriptions, etc.) associated with the user 20. The user 20 may opt in to allow the web server 50 and/or the communication device 10 to access the user's medical records and obtain the medical indicia from the medical record processing facility 90. Also, once the customized cosmeceutical formulation has been made, the formula can be added to the medical record for the user 20 to maintain evidence of the user having ordered the customized cosmeceutical formulation, thereby to facilitate future diagnoses and interventions by medical practitioners having access to the user's medical record.

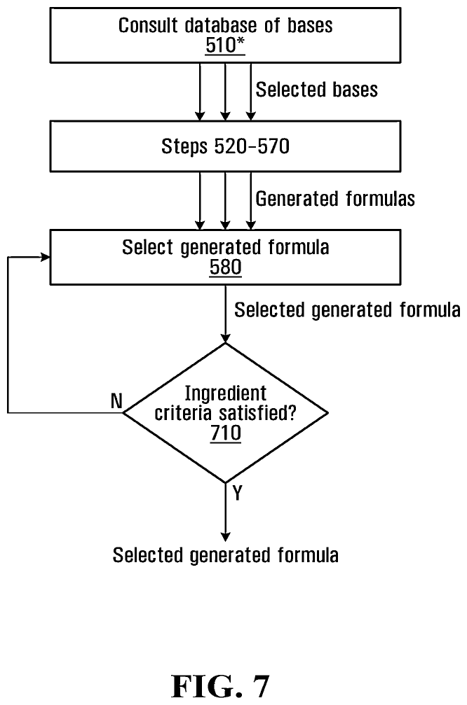

According to a further enhancement, the compounding facility 40 (e.g., a compounding pharmacy or a network of compounding pharmacies) stores expiry data information about batches of the ingredients stocked at the compounding facility 40. For example, and with reference to FIG. 10, the networked computer at the compounding facility 40 may include a database conceptually shown at 1000, which maintains a record of the quantities and expiry dates of various bases and boosters or the ingredients used by such bases and boosters. It is seen that there may be plural batches of the same base (e.g., Base 1) or of the same booster (e.g., Booster C), with different quantities and/or expiry dates.

In this embodiment, the formulation rules engine 284 generates the generated formula in a unique way. Specifically, generation of the generated formula may be subjected to constraints on the expiry dates of batches of the various ingredients stocked at the compounding facility 40. For example, batches of ingredients that are sooner to expire (as indicated by the expiry dates in the database 1000) may be prioritized by the cosmeceutical utility 280 over batches ingredients that are later to expire.

For example, consider a generated formula that needs Base 1 and Boosters A and B, where Booster C was not selected but remains part of the original array of candidate boosters. In this case, upon realizing that Booster B has an expiry date that is much later than Booster C (e.g., more than a threshold number of months, such as 3 of 6 months-in the example of FIG. 10, the 75 mL of Booster C expires 4 months before Booster B), the cosmeceutical utility 280 removes Booster B from the formula and swaps in Booster C. Of course, the cosmeceutical utility 280 may be configured to test whether the resulting (revised) formula is pre-tested and, if so, the generated formula will include Base 1 with Boosters A and C, which may not have been considered optimal but is still capable of satisfying the expressed needs or constraints of the user 20 (the skincare profile), yet is more commercially advantageous for the compounding facility 40 than the combination of Base 1 with Boosters A and B.

In the case where there are several generated formulas from which one is selected by the formula rules engine 284, this could affect which formula is actually the one selected. This could result in a different generated formula being selected under the aforementioned constraints on the expiry dates than in the absence of such constraints. For example, where there is a first generated formula and a second generated formula, the formulation rules engine 284 may be configured to identify the earliest expiry date of the ingredients needed to produce the first generated formula and the earliest expiry date of the ingredients needed to produce the second generated formula, and to select, as the selected generated formula, the one generated formula with the earlier of the two earliest expiry dates.

In a variant, the above concept is expanded to a distributed compounding environment with multiple compounding pharmacies that may be eligible for carrying out the compounding for a particular user (e.g., the user 20). These compounding pharmacies each store expiry date information about the batches of ingredients that they each stock, i.e., each maintains a database such as the database 1000. Alternatively a central database is held, and the database includes an additional field for each ingredient batch indicative of the pharmacy that stocks that particular batch. In this variant, constraints on the expiry dates may impact the selection of (i) a generated formula as well as (ii) the actual compounding pharmacy where the customized cosmeceutical formulation is to be compounded. In fact, even if there is only one generated formula from which to choose (e.g., FIG. 5A), there may be provided the added step of selecting of the compounding pharmacy that will be tasked with production of the customized cosmeceutical formulation.

For example, consider a generated formula that needs Base 1 and Boosters A and B. Consider also that pharmacies P1 and P2 are part of a common pharmacy network and each stock adequate quantities of Base 1 and Boosters A and B. However, Booster A stocked at pharmacy P1 expires in 6 months and Booster A stocked at pharmacy P2 expires in 9 months, whereas Booster B stocked at pharmacy P1 expires 3 months and Booster B stocked at pharmacy P2 expires in 8 months. In this case, the upon realizing that Booster B stocked at pharmacy P1 is the first to expire, the cosmeceutical utility 280 selects P1 as the pharmacy of choice for compounding the generated formula. Pharmacy P1 may be further from, or less convenient than, pharmacy P2 for the user, but may provide a better commercial outcome for the owner of the pharmacy network. In other embodiments, the total number of months until expiration (of all ingredients) may be the expiry-date-related metric used to select the pharmacy where compounding of the generated formula will take place.

Of course, a combination of the above features is possible, whereby the expiry dates and available quantities of the bases and boosters, as well as the expiry dates and available quantities of the individual chemicals that make up the bases and boosters, may be combinedly processed so as to make a decision about where to produce a formulation based on a generated formula, as well as the composition of the generated formula itself.

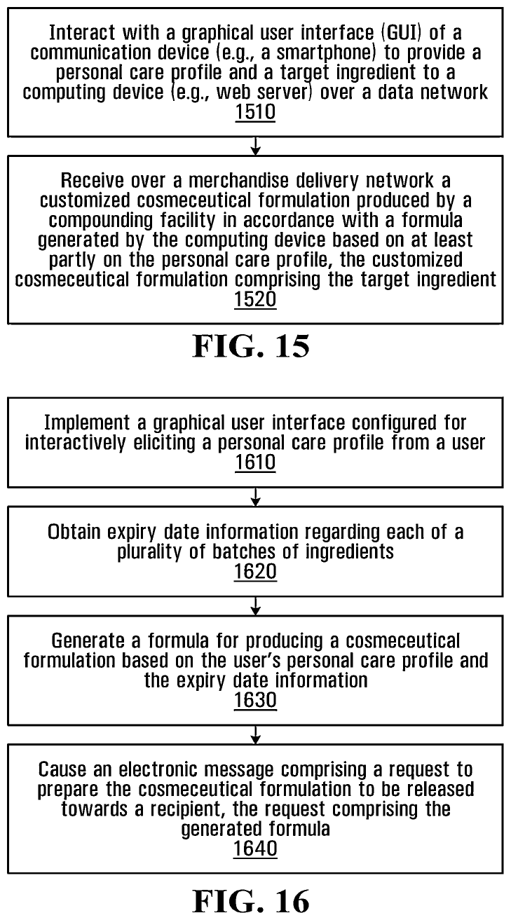

As such, there has been provided a computer-implemented method, which is depicted in the flowchart of FIG. 16 as comprising the steps of implementing a graphical user interface configured for interactively eliciting a personal care profile from a user (step 1610); obtaining expiry date information regarding each of a plurality of batches of ingredients (step 1620); generating a formula for producing a cosmeceutical formulation based on the user's personal care profile and the expiry date information (step 1630); and causing an electronic message comprising a request to prepare the cosmeceutical formulation to be released towards a recipient, the request comprising the generated formula (step 1640).

While the above embodiments were described primarily in the context of skin care, other embodiments may be relevant to other cosmetic fields, including hair care.

1-34. (canceled)

35. A system for initiating preparation of a customized cosmeceutical formulation, comprising:
a non-transitory storage medium operably storing computer-readable program instructions; and
at least one processor connected to the non-transitory storage medium and configured to execute the program instructions, wherein execution of the program instructions by the at least one processor causes instantiation of a cosmeceutical utility configured for:
implementing a graphical user interface configured for interactively eliciting input from a user to define a personal care profile for the user;
generating a plurality of formulas for producing a plurality of cosmeceutical formulations based on the personal care profile;
obtaining expiry date information regarding each of a plurality of batches of ingredients;
determining an earliest expiry date for a first formula of the two or more of the plurality of formulas based on the expiry date information;
determining an earliest expiry date for a second formula of the two of more of the plurality of formulas based on the expiry date information;
selecting, between the first and second formulas, the formula having the earliest expiry date of the respective earliest expiry dates; and
causing an electronic message comprising a request to prepare the cosmeceutical formulation to be released towards a recipient, the request comprising the selected formula.

36. The system of claim 35, wherein generating the plurality of formulas comprises consulting a database of formulas to identify a particular one of the formulas that at least partly satisfies the personal care profile, wherein the cosmeceutical utility is configured for prioritizing inclusion of sooner-to-expire batches of ingredients in the generated formula over the inclusion of later-to-expire batches of ingredients.

37. The system of claim 35, wherein the recipient is a selected compounding pharmacy of a plurality of compounding pharmacies, and wherein the cosmeceutical utility is further configured for obtaining an identifier of a particular one of the compounding pharmacies where each of the batches of ingredients is stored.

38. The system of claim 35, wherein generating the plurality of formulas comprises:
consulting a database of formulas to identify a particular formula that at least partly satisfies the personal care profile;
identifying ingredients in the particular formula;
determining expiry dates of the identified ingredients; and
substituting at least one ingredient of the identified ingredients with a substitute ingredient that has an expiry date later than an expiry date of the at least one ingredient.

39. The system of claim 35, wherein generating the plurality of formulas comprises:
consulting a database of formulas to identify a plurality of formulas that at least partly satisfy the personal care profile;
identifying expiry dates associated with the ingredients in the plurality of formulas; and
selecting the formula having earliest expiry dates of the ingredients as the generated formula from among the identified plurality of formulas.

40. The system of claim 36, wherein the database maintains a record of quantities and the expiry dates associated with the ingredients in the plurality of formulas.

41. The system of claim 37, wherein the selected compounding pharmacy is subject to another constraint that the selected compounding pharmacy stores an ingredient having an earliest expiry, the ingredient being one of the ingredients in the formula.

42. The system of claim 35, wherein the recipient includes a mixer device to prepare the cosmeceutical formulation at least in part, the request including mixing parameters that are extracted by a networked computer and supplied to a mixing device to prepare the cosmeceutical formulation.

43. A method, comprising:
implementing a graphical user interface configured for interactively eliciting a personal care profile from a user;
obtaining expiry date information regarding each of a plurality of batches of ingredients;
generating a formula for producing a cosmeceutical formulation based on the personal care profile and the expiry date information;
causing an electronic message comprising a request to prepare the generated formula towards a recipient, the request comprising the generated formula; and
at the recipient, receiving the generated formula and compounding the cosmeceutical formulation according to the generated formula.

44. The method of claim 43, wherein generating the formula comprises consulting a database of formulas to identify a particular one of the formulas that at least partly satisfies the personal care profile, wherein cosmeceutical utility is configured for prioritizing inclusion of sooner-to-expire batches of ingredients in the generated formula over the inclusion of later-to-expire batches of ingredients.

45. The method of claim 43, further comprising:
identifying a plurality of compounding pharmacies where each of the batches of ingredients is stored; and
selecting a particular compounding pharmacy from the plurality of compounding pharmacies based on a constraint that the selected particular compounding pharmacy stores a sufficient quantity of all the ingredients in the formula.

46. The method of claim 45, wherein selecting the particular compounding pharmacy is subject to an additional constraint that the selected compounding pharmacy stores an ingredient having an earliest expiry, the ingredient being one of the ingredients in the formula.

47. The method of claim 43 wherein generating the formula comprises:
consulting a database of formulas to identify a particular formula that at least partly satisfies the personal care profile;
identifying ingredients in the particular formula;
determining expiry dates of the identified ingredients; and
substituting at least one ingredient of the identified ingredients with a substitute ingredient that has an expiry date later than an expiry date of the at least one ingredient.

48. The method of claim 43, wherein generating the formula comprises:
consulting a database of formulas to identify a plurality of formulas that at least partly satisfy the personal care profile;
identifying expiry dates of the ingredients in the plurality of formulas; and
selecting the formula having earliest expiry dates of the ingredients as the generated formula from among the identified plurality of formulas.

49. The method of claim 48, further comprising
maintaining, in the database, a record of quantities and the expiry dates associated with the ingredients in the plurality of formulas.
